(19) United States
(12) Patent Application Publication
(10) Pub. No.: US 2026/0166269 A1
(43) Pub. Date: Jun. 18, 2026

(54) INTERMITTENT URINARY CATHETER

(71) Applicant: ConvaTec Limited, Flintshire (GB)

(72) Inventors: Kate Duhig, Chester (GB); Marian Novak, Trebisov (SK); Nicola Pilmer, Chester (GB); Daniel Allen, Staffordshire (GB); Gavin Roberts, Swindon (GB)

(21) Appl. No.: 19/536,415

(22) Filed: Feb. 11, 2026

Related U.S. Application Data
(63) Continuation of application No. PCT/GB2024/052107, filed on Aug. 9, 2024.
(60) Provisional application No. 63/518,918, filed on Aug. 11, 2023.

(30) Foreign Application Priority Data
Sep. 11, 2023 (GB) 2313805.0

(51) Int. Cl. A61M 25/00; A61M 25/01; A61M 25/09

(52) U.S. Cl. CPC A61M 25/007; A61M 25/0017; A61M 25/002; A61M 25/0023; A61M 25/01; A61M 25/09; A61M 2025/0059; A61M 2210/1085

(57) ABSTRACT

An intermittent urinary catheter comprises an elongate main body. The elongate main body has an insertion end for insertion into a urethra and bladder, an outlet end for the outflow of fluid from the catheter, and a lumen extending between the insertion end and the outlet end. The elongate main body also comprises at least one inlet aperture proximal to the insertion end, and an external flushing portion extending along a portion of the elongate main body between the insertion end and the outlet end. The external flushing portion comprises a plurality of elongate eyelets formed through an external surface of the elongate main body.

### TECHNICAL FIELD OF THE INVENTION

The present invention relates to an intermittent urinary catheter which is configured to aid flushing of the urethra in use.

### BACKGROUND TO THE INVENTION

Urinary catheters for draining urine from the bladder may be indwelling or intermittent and are used to drain urine from a bladder. Intermittent catheters are typically used by patients suffering from urinary incontinence or by disabled individuals several times a day to drain the bladder as required.

It is relatively common for users of intermittent catheters to develop urinary tract infections or other complications. In some cases, this is because intermittent catheters typically comprise a closed lumen which expels urine from the bladder without it contacting the urethra wall. As such, there can be a persistent build-up of bacteria and debris (e.g., skin cells or excretions) which would otherwise be flushed out by the urine.

U.S. Pat. No. 1,045,326A discloses an irrigating catheter comprising a flexible tubular body of substantially uniform external diameter from end to end, said body provided externally adjacent one of its ends with an annular groove depressed below the general outer surface of said body and extending around said body, said body also provided externally with similarly depressed, longitudinal grooves leading from said annular groove and extending throughout the greater portion of the length of said body and said body also provided intermediate the ends of said longitudinal grooves with apertures leading from the bottom of said annular groove into the internal cavity of the catheter.

U.S. Pat. No. 10,668,249B2 discloses a catheter provided with a distal portion, a proximal portion, and a plurality of drainage eyes disposed at a junction between the distal portion and the proximal portion. The distal portion includes a tip, a plurality of elongated ribs, and a plurality of external flow paths. The proximal portion includes an internal lumen and a proximal end. The plurality of drainage eyes communicate with the plurality of external flow paths and the internal lumen.

The present invention seeks to provide an improved intermittent urinary catheter for flushing the urethra in use.

### SUMMARY OF THE INVENTION

The present disclosure provides an intermittent urinary catheter according to the appended claims.

The present disclosure provides, in a broad aspect, a catheter. The catheter may be a urinary catheter. The catheter may be an intermittent urinary catheter. The intermittent urinary catheter may comprise an elongate main body. The elongate main body may have an insertion end. The insertion end may be for insertion into a urethra and bladder. The elongate main body may have an outlet end. The outlet end may be for the outflow of fluid from the catheter. The elongate main body may have a lumen. The lumen may extend between the insertion end and the outlet end. The elongate main body may further comprise at least one inlet aperture. The at least one inlet aperture may be proximal to the insertion end. The elongate main body may further comprise an external flushing portion. The external flushing portion may extend along a portion of the elongate main body between the insertion end and the outlet end. The external flushing portion may comprise a plurality of elongate eyelets. The plurality of elongate eyelets may be formed through an external surface of the elongate main body.

The present disclosure provides, in a first aspect, an intermittent urinary catheter comprising: an elongate main body having an insertion end for insertion into a urethra and bladder, an outlet end for the outflow of fluid from the catheter, and a lumen extending between the insertion end and the outlet end, the elongate main body further comprising at least one inlet aperture proximal to the insertion end, and an external flushing portion extending along a portion of the elongate main body between the insertion end and the outlet end, wherein the external flushing portion comprises a plurality of elongate eyelets formed through an external surface of the elongate main body.

Providing an external flushing portion comprising a plurality of elongate eyelets formed through the external surface of the elongate main body is particularly advantageous as it allows a flow of urine to contact the urethra during use. The flow of urine may act to flush the urethra and help reduce the possibility of infection. Furthermore, providing the external flushing portion with a plurality of elongate eyelets provides a relatively large amount of space through which urine may flow externally from the catheter to contact and flush the urethra in use whilst also providing a resilient structure capable of supporting and maintaining the urethra in an open state without warping or buckling under the pressures exerted by the wall of the urethra. Catheter arrangements having external channels along the outside of the main body which act to flush the urethra during use have not found favour with users. This is likely because users do not like the idea of having a flow of urine down an external surface of the catheter which they believe may be unnecessarily messy. Therefore, providing an intermittent urinary catheter with a plurality of elongate eyelets is more appealing to users than providing alternative flushing features such as long channels, as eyelets are commonly found in intermittent urinary catheters (although hitherto, only as inlets), making such an intermittent urinary catheter more easily recognisable as a standard catheter. Therefore, users are not as deterred by the eyelets and feel much more comfortable using such a catheter, whilst still receiving the benefits of flushing of the urethra.

There may be provided any suitable number of inlet apertures. There may be provided between one and four inlet apertures. In a preferred embodiment, the elongate main body may comprise two (and optionally only two) inlet apertures. The two inlet apertures may be circumferentially offset. For example, the two inlet apertures may be diametrically opposed. The two inlet apertures may be longitudinally offset. For example, the inlet apertures may be longitudinally offset and diametrically opposed. Providing the two inlet apertures diametrically opposed enables the bladder to be emptied from both sides of the catheter without compromising the structural integrity of the catheter. Furthermore, providing longitudinally offset inlet apertures enables the catheter to be inserted into the bladder by varying amounts without compromising or restricting flow of urine into the lumen.

The at least one inlet aperture may be configured to be fully inserted into the bladder. Additionally or alternatively, the at least one inlet portion may be configured to be partially inserted into the bladder. This configuration is particularly advantageous in ensuring full bladder emptying as the at least one inlet aperture overlaps both the urethra and bladder ensuring that any urine residing on the bottom of the bladder may be emptied via the at least one inlet aperture.

The at least one inlet aperture may comprise a means for directing flow. The at least one inlet aperture may comprise an open flow channel. The open flow channel may be provided in the external surface of the elongate main body. The open flow channel may extend over at least one quarter of the length of the at least one inlet aperture. The open flow channel may extend over at least one third of the length of the at least one inlet aperture. The open flow channel may extend over at least one half of the length of the at least one inlet aperture. The open flow channel may comprise a wall. The wall may be recessed from an external surface of the elongate main body.

Providing an inlet aperture having an open flow channel is particularly advantageous in directing urine from the bladder, through the at least one inlet aperture to the lumen such that the bladder may be efficiently emptied. The urine may then pass through the lumen and out of the catheter via the external flushing portion.

The open flow channel may comprise a base. The base may be substantially flat. Alternatively, the base may have a curved profile. Additionally or alternatively, the open flow channel may comprise a pair of side walls. The pair of side walls may be substantially flat. Additionally or alternatively, the pair of side walls may have a curved profile. The pair of side walls may be provided adjacent to the base. For example, one of the pair of side walls may extend along the left hand side of the base and the other side wall may extend along the right hand side of the base. The pair of side walls may be angled to form the open flow channel with a substantially “V” or “U” shaped cross section. The cross-sectional area of the open flow channel may be constant along a major portion of the length thereof. The cross-sectional area of the open flow channel may be constant along at least half of the length thereof. Providing an open flow channel having a cross section as described herein ensures a constant and efficient flow of urine through the open flow channel without being disturbed by irregularities within the cross section.

Additionally or alternatively, the open flow channel may further comprise an angled portion. The angled portion may be provided at the proximal end of the open flow channel. The angled portion may be configured to direct fluid from the bladder toward the lumen. There may be provided an insertion portion. The insertion portion may facilitate insertion of the catheter into and along the urethra. The insertion portion may be hollow. The insertion portion may comprise a rounded tip. The rounded tip may be provided at the insertion end. The rounded tip may aid insertion of the catheter into the bladder via the urethra. Providing a rounded tip improves comfort of the user, particularly during insertion of the catheter into the urethra. The insertion portion may comprise a hollow tubular wall. The hollow tubular wall may define the lumen. The at least one inlet aperture may be provided in the hollow tubular wall. The at least one open flow channel may be integrally formed with the hollow tubular wall. The base and/or pair of side walls may be integrally formed with the hollow tubular walls. The at least one inlet aperture may be in fluid communication with the lumen. The at least one inlet aperture may be in fluid communication with the external flushing portion via the lumen.

Providing at least one inlet aperture in fluid communication with the lumen allows for a more complete emptying of the bladder and flushing of the urethra via the external flushing portion.

The at least one inlet aperture may be rectangular. The at least one inlet aperture may be oval. In a preferred embodiment, the at least one inlet aperture may be stadium shaped. A stadium shaped inlet aperture is particularly advantageous as it does not comprise any pointed corners which may catch the walls of the urethra during insertion and removal of the catheter causing discomfort for the user.

The at least one inlet aperture may comprise an angled edge, or ‘chamfer’. The angled edge may extend partially or fully around the perimeter of the at least one inlet aperture. In a preferred embodiment, the angled edge may extend fully around the perimeter of the at least one inlet aperture. Providing an angled edge is particularly advantageous in increasing user comfort during insertion and use as it minimises the risk of sharp edges catching and cutting the users skin. The angled edge may be straight. The angled edge may be curved. The angled edge may be convex i.e. ‘radiused’. The angled edge may be concave.

The at least one inlet aperture may have a width in the range of 0.8 mm to 1.2 mm. The at least one inlet aperture may have a width in the range of 0.85 mm to 1.15 mm. The at least one inlet aperture may have a width in the range of 0.9 mm to 1.1 mm. The at least one inlet aperture may have a width in the range of 0.95 mm to 1.05 mm. In a preferred embodiment, the at least one inlet aperture may have a width of 1 mm.

The at least one inlet aperture may have a length in the range of 5 mm to 16 mm. The at least one inlet aperture may have a length in the range of 9 mm to 15.5 mm. The at least one inlet aperture may have a length in the range of 13 mm to 15 mm. In a preferred embodiment, the at least one inlet aperture may have a length of 14 mm.

The elongate main body may be straight (i.e., in the absence of bending forces, at rest, the elongate main body may be substantially straight).

The external surface of the elongate main body may form a hollow tubular wall. The hollow tubular wall may be substantially cylindrical. The hollow tubular wall may define the lumen. The lumen may be a hollow passageway. The lumen may extend from the insertion portion to the outlet end. The lumen may be in fluid communication with the plurality of elongate eyelets. The lumen may define a fluid flow path from the insertion portion to the outlet end. The lumen may comprise a first end and a second end. The first end may be in fluid communication with the at least one inlet aperture. The second end may be provided at the outlet end. Urine may exit the lumen and therefore the catheter at the second end. The lumen may have constant cross section along the length thereof. Alternatively, the lumen may have a constant cross section along a major portion of the length thereof. For example, the lumen may have a constant cross section along at least 70%, 80% or 90% of the length thereof.

The external flushing portion may be configured, in use, to provide fluid communication between the catheter and the urethra via the plurality of elongate eyelets. Providing fluid communication between the catheter and the urethra via the plurality of elongate eyelets is particularly advantageous as it allows a flow of urine to contact the walls of the urethra during use, helping to reduce the possibility of infection.

The plurality of elongate eyelets may have a longitudinal axis. The longitudinal axis of the plurality of elongate eyelets may extend parallel to the longitudinal axis of the main body. The plurality of elongate eyelets may be spaced along the longitudinal axis of the elongate main body. The plurality of elongate eyelets may be spaced by a fixed distance along the longitudinal axis of the elongate main body. The plurality of elongate eyelets may be at least partially spaced along the longitudinal axis of the elongate main body. At least one of the plurality of elongate eyelets may be at least partially spaced along the longitudinal axis of the elongate main body. The plurality of elongate eyelets may be completely spaced along the longitudinal axis of the elongate main body. At least one of the plurality of elongate eyelets may be completely spaced along the longitudinal axis of the elongate main body. The plurality of elongate eyelets may be spaced by the length of at least one of the plurality of elongate eyelets. Providing the plurality of elongate eyelets completely spaced is particularly advantageous as it provides an improved fluid flow path for urine passing through the lumen having a more constant cross section which minimises turbulence of the flow passing through. Additionally or alternatively, at least one or each of the plurality of elongate eyelets may be spaced by the same distance along the longitudinal axis of the elongate main body. Additionally or alternatively, at least one or each of the plurality of elongate eyelets may be spaced by a different distance along the longitudinal axis of the elongate main body. Providing the plurality of elongate eyelets spaced along the longitudinal axis of the elongate main body is particularly advantageous as it ensures that the structural integrity of the catheter is maintained such that it may resist bending and buckling forces exerted during use whilst allowing flushing of the urethral wall which may prevent the build-up of bacteria. The spacing of the plurality of elongate eyelets provides flexibility which is particularly advantageous during insertion of the catheter as it enables the catheter to adapt to the shape of the urethra into which it is being inserted.

At least one of the plurality of elongate eyelets may not directly oppose any other of the plurality of the elongate eyelets.

In alternative embodiments, at least one of the plurality of elongate eyelets may directly oppose another of the plurality of elongate eyelets. Additionally or alternatively, at least one of the plurality of elongate eyelets may partially oppose another of the plurality of elongate eyelets.

The plurality of elongate eyelets may be provided along a plane of the longitudinal axis of the elongate main body. A first series of elongate eyelets may be formed through a first side of the elongate main body. A second series of elongate eyelets may be formed through a second side of the elongate main body. Each series may comprise any suitable number of elongate eyelets. One or each series may comprise at least two elongate eyelets. One or each series may comprise at least three elongate eyelets. One or each series may comprise at least four elongate eyelets. One or each series may comprise at least five elongate eyelets. One or each series may comprise at least six elongate eyelets. One or each series may comprise at least seven elongate eyelets. One or each series may comprise at least eight elongate eyelets. One or each series may comprise at least nine elongate eyelets. One or each series may comprise at least ten elongate eyelets. Each of the first series and second series may comprise the same number of elongate eyelets. Each of the first series and second series may comprise a different number of elongate eyelets. In a preferred embodiment, each series may comprise three elongate eyelets. In a preferred embodiment, the first series of three elongate eyelets may be formed through the first side of the elongate main body and the second series of three elongate eyelets may be formed through the second side of the elongate main body. The first side of the elongate main body and the second side of the elongate main body may be diametrically opposed. The eyelets of the first series may be staggered with respect to the eyelets of the second series such that at least one eyelet of the first series opposes the space between eyelets of the second series. The pitch of the staggered eyelets may be such that no part of an eyelet of the first series directly opposes any part of an eyelet of the second series.

None of the elongate eyelets of the first series may directly oppose an elongate eyelet of the second series. Providing the series of elongate eyelets in this particular arrangement is particularly advantageous for manufacturing the catheters via injection moulding as the plurality of elongate eyelets do not extend through both a first side and diametrically opposing second side at the same point along the length of the elongate main body. This also helps to maintain rigidity and resistance to bending and/or buckling of the catheter during use.

In an alternative embodiment, the first series of elongate eyelets (e.g. the first series of three elongate eyelets) may be formed through the first side of the elongate main body and the second series of elongate eyelets (e.g. the second series of three elongate eyelets) may be formed through the second side of the elongate main body. The first series of elongate eyelets and the second series of elongate eyelets may be diametrically opposed. At least one of the elongate eyelets of the first series may directly oppose any of the elongate eyelets of the second series. Each of the elongate eyelets of the first series may directly oppose an elongate eyelet of the second series.

The plurality of elongate eyelets may have a substantially rectangular shape, for example, rectangular with rounded corners. The plurality of elongate eyelets may have an oval shape. In a preferred embodiment, the plurality of elongate eyelets may have a stadium shape. The plurality of elongate eyelets may comprise a pair of long sides. The long sides may be parallel. The long sides may extend longitudinally. The pair of long sides may be connected by a first short side and a second short side. At least one of the first and second short sides may be straight. At least one of the first and second short side may be curved. In a preferred embodiment, both the first and second short sides are curved. Providing at least one curved short side is particularly advantageous during insertion and withdrawal of the catheter into the urethra as it minimises the possibility of sharp edges catching and/or cutting the walls of the urethra.

At least one of the pair of long sides and/or the first short side and/or the second short side may comprise an angled edge, or ‘chamfer’. In a preferred embodiment all the sides comprise an angled edge, thereby minimising the presence of sharp edges cutting or catching the urethral wall. The angled edge may be straight. The angled edge may be curved. The angled edge may be convex i.e. ‘radiused’. The angled edge may be concave.

At least one of the plurality of elongate eyelets may have a width of at least 0.7 mm. At least one of the plurality of elongate eyelets may have a width of at least 0.8 mm. At least one of the plurality of elongate eyelets may have a width of at least 0.85 mm. At least one of the plurality of elongate eyelets may have a width of at least 0.9 mm. At least one of the plurality of elongate eyelets may have a width of at least 0.95 mm. At least one of the plurality of elongate eyelets may have a width of at least 1 mm. At least one of the plurality of elongate eyelets may have a width of at least 1.05 mm. At least one of the plurality of elongate eyelets may have a width of at least 1.1 mm. At least one of the plurality of elongate eyelets may have a width of at least 1.15 mm. At least one of the plurality of elongate eyelets may have a width of at least 1.2 mm. At least one of the plurality of elongate eyelets may have a width of at least 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, or 2.5 mm. At least one of the plurality of elongate eyelets may have a width of no more than 2.5 mm, 2.4 mm, 3.3 mm, 2.2 mm, 2.1 mm, 2 mm, 1.9 mm, 1.8 mm, 1.7 mm, 1.6 mm, 1.5 mm, 1.4 mm, 1.3 mm, 1.2 mm, 1.1 mm, 1.15 mm, 1.1 mm, or 1.05 mm. At least one of the plurality of elongate eyelets may have a width in the range of 0.7 mm to 2.5 mm. At least one of the plurality of elongate eyelets may have a width in the range of 0.8 mm to 1.2 mm. At least one of the plurality of elongate eyelets may have a width in the range of 0.85 mm to 1.15 mm. At least one of the plurality of elongate eyelets may have a width in the range of 0.9 mm to 1.1 mm. At least one of the plurality of elongate eyelets may have a width in the range of 0.95 mm to 1.05 mm. In a preferred embodiment, at least one of the plurality of elongate eyelets may have a width of 1 mm.

At least one of the plurality of elongate eyelets may have a length of at least 2.5 mm. At least one of the plurality of elongate eyelets may have a length of at least 3 mm. At least one of the plurality of elongate eyelets may have a length of at least 3.5 mm. At least one of the plurality of elongate eyelets may have a length of at least 4 mm. At least one of the plurality of elongate eyelets may have a length of at least 4.5 mm. At least one of the plurality of elongate eyelets may have a length of at least 5 mm. At least one of the plurality of elongate eyelets may have a length of at least 5.5 mm. At least one of the plurality of elongate eyelets may have a length of at least 6 mm. At least one of the plurality of elongate eyelets may have a length of at least 6.5 mm. At least one of the plurality of elongate eyelets may have a length of at least 7 mm. At least one of the plurality of elongate eyelets may have a length of at least 7.5 mm. At least one of the plurality of elongate eyelets may have a length of at least 8 mm. At least one of the plurality of elongate eyelets may have a length of at least 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, or 25 mm. At least one of the plurality of eyelets may have a length no more than 30 mm, 25 mm, 20 mm, 15 mm, 14 mm, 13 mm, 12 mm, 11 mm, 10 mm, 9 mm, 8 mm, or 7 mm. At least one of the plurality of elongate eyelets may have a length in the range of 2.5 mm to 25 mm. At least one of the plurality of elongate eyelets may have a length in the range of 2.5 mm to 8 mm. At least one of the plurality of elongate eyelets may have a length in the range of 4.5 mm to 7.75 mm. At least one of the plurality of elongate eyelets may have a length in the range of 6.5 to 7.5 mm. In a preferred embodiment, at least one of the plurality of elongate eyelets may have a length of 7 mm.

The external flushing portion may comprise at least 10% of the length of the catheter. The external flushing portion may comprise at least 20% of the length of the catheter. The external flushing portion may comprise at least 30% of the length of the catheter. The external flushing portion may comprise at least 40% of the length of the catheter. The external flushing portion may comprise at least 50% of the length of the catheter. The external flushing portion may comprise at least 60% of the length of the catheter. The external flushing portion may comprise at least 70% of the length of the catheter. The external flushing portion may comprise at least 80% of the length of the catheter. The external flushing portion may comprise at least 90% of the length of the catheter. The external flushing portion may comprise between 10% and 90% of the length of the catheter. The external flushing portion may comprise between 20% and 80% of the length of the catheter. The external flushing portion may comprise between 30% and 70% of the length of the catheter.

The elongate main body may have a constant outer diameter. The insertion portion and external flushing portion may have the same outer diameter. Providing the elongate main body with a constant outer diameter is particularly advantageous as it improves comfort for the user during insertion and withdrawal of the catheter.

The catheter may be formed as a singular piece. Alternatively, the catheter may be formed from separate component pieces that are attached together. The catheter may be formed via injection moulding. Alternatively, the catheter may be formed via extrusion. Additionally or alternatively, the plurality of elongate eyelets and the at least one inlet aperture may be formed by punching.

The outlet end may be shaped for the attachment of a separate funnel. The outlet end may be configured for the attachment of separate funnel. The outlet end may be attached to a funnel. The outlet end may be formed integrally with a funnel. The funnel may comprise an aperture therethrough to enable the throughflow of fluid.

The outlet end may comprise the second end of the lumen. The outlet end may be open. The outlet end may facilitate the outflow of fluid from the catheter. The outlet end may guide fluid from the catheter into a suitable receptacle such as a toilet bowl.

An outlet portion may be provided. The outlet portion may not comprise any apertures and/or elongate eyelets. The outlet portion may be provided toward the outlet end. In a preferred embodiment, the outlet portion may terminate at the outlet end. The outlet portion may comprise a solid tubular wall. The outlet portion may be configured for at least partial insertion into the urethra. Providing an enclosed solid, tubular outlet portion is particularly advantageous as it ensures that the flow of urine is contained within the outlet portion and does not leak out.

The outlet end may be provided by an opening in an end face of the catheter. The second end of the lumen may be provided by the opening in the end face of the catheter.

The catheter may be a male catheter or a female catheter. In a preferred embodiment, the catheter is a female intermittent urinary catheter.

The length of the insertion portion may be between 20 mm and 50 mm. Preferably, the length of the insertion portion may be 30 mm.

The length of the outlet portion may be between 10 mm and 30 mm. Preferably the length of the outlet portion may be 20 mm.

For a female catheter. The length of the catheter may be between 80 mm and 140 mm, preferably 100 mm. The length of the external flushing portion may be at least 60 mm. The length of the external flushing portion may be at least 70 mm. The length of the external flushing portion may be at least 80 mm. The length of the external flushing portion may be at least 90 mm. The length of the external flushing portion may be at least 100 mm. The length of the external flushing portion may be at least 110 mm. The length of the external flushing portion may be at least 120 mm. The length of the external flushing portion may be between 60 mm and 120 mm. In a preferred embodiment, the length of the external flushing portion may be 100 mm.

For a male catheter, the length of the catheter may be between 360 mm and 425 mm, preferably 405 mm. The length of the external flushing portion may be between 300 mm and 350 mm, preferably 325 mm.

The diameter of the insertion portion and the external flushing portion may be between 2 mm and 6 mm depending on the size requirements of the intended user.

The depth of the at least one flushing channel may be between 0.5 mm and 2 mm, preferably 1 mm.

The present disclosure provides, in a second aspect, a method of manufacturing an intermittent urinary catheter comprising the steps of: providing an elongate main body having at least one inlet aperture and a lumen; and forming a plurality of elongate eyelets through an external surface of the elongate main body.

The method of manufacturing an intermittent urinary catheter may comprise the steps of injection moulding the elongate main body having at least one inlet aperture and a lumen and a plurality of elongate eyelets through an external surface of the elongate main body.

Alternatively, the method of manufacturing an intermittent urinary catheter may comprise the steps of extruding the elongate main body comprising the lumen and punching the at least one inlet aperture and the plurality of elongate eyelets through an external surface of the elongate main body.

Additionally or alternatively, the method of manufacturing an intermittent urinary catheter may further comprise the steps of providing a funnel and attaching the funnel to an outlet end of the elongate main body.

Additionally or alternatively, the method of manufacturing an intermittent urinary catheter may further comprise the steps of integrally forming the funnel at the outlet end of the elongate main body.

The present disclosure provides, in a third aspect, a method of catheterisation using a catheter according to the first aspect of the invention, optionally including any optional features thereof. The method may comprise the steps of: (i) providing an intermittent urinary catheter of the first aspect, and (ii) inserting the catheter into the bladder via the urethra until the external flushing portion is in fluid communication with the bladder so that urine can drain from the bladder through the urethra to the outlet end via the plurality of elongate eyelets of the external flushing portion.

The person skilled in the art will appreciate that except where mutually exclusive, a feature described in relation to any one of the aspects, embodiments or examples described herein may be applied to any other aspect, embodiment or example. Furthermore, except where mutually exclusive, any feature described herein may be applied to any aspect and/or combined with any other feature described herein.

### DETAILED DESCRIPTION OF THE INVENTION

In order that the invention may be more clearly understood one or more embodiments thereof will now be described, by way of example only, with reference to the accompanying drawings, of which:

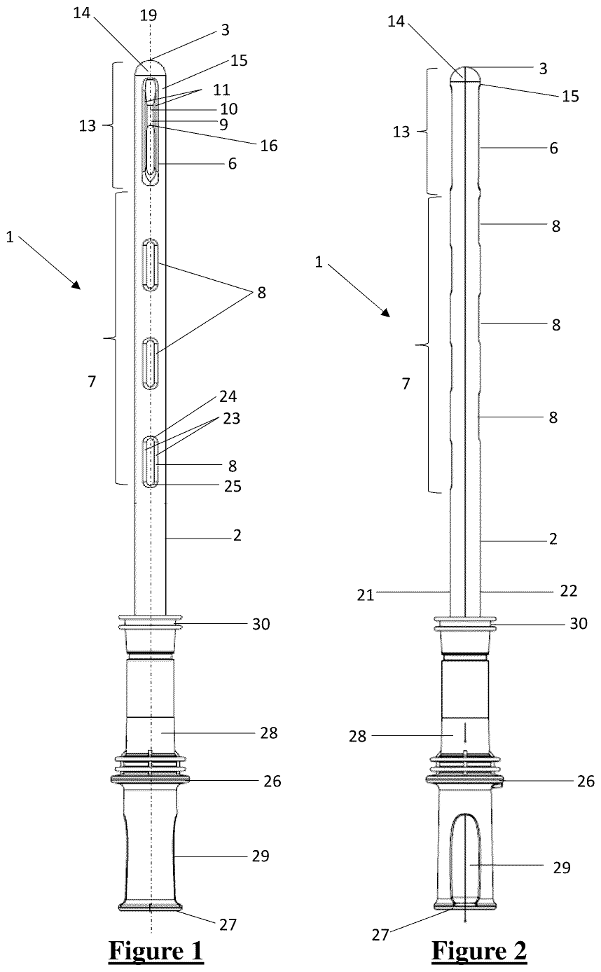

FIG. 1 shows a side view of a catheter according to the present disclosure;

FIG. 2 shows a side view of the catheter of FIG. 1 at 90° to the view of FIG. 1;

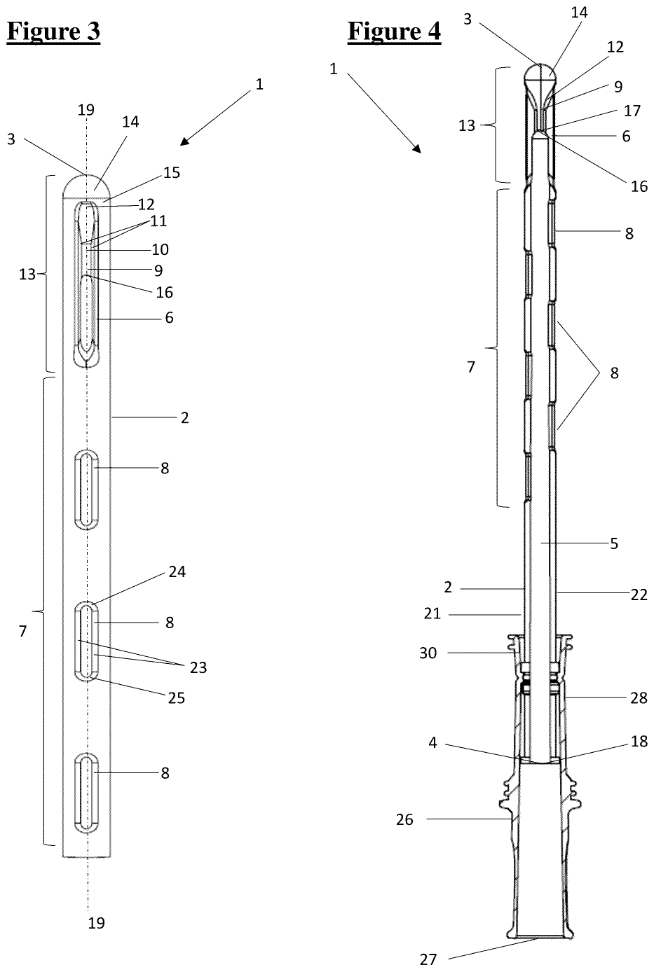

FIG. 3 shows an enlarged side view of the inlet apertures and plurality of elongate eyelets of the catheter from the angle of FIG. 1;

FIG. 4 shows a longitudinal sectional side view of the catheter from the same angle of FIG. 2;

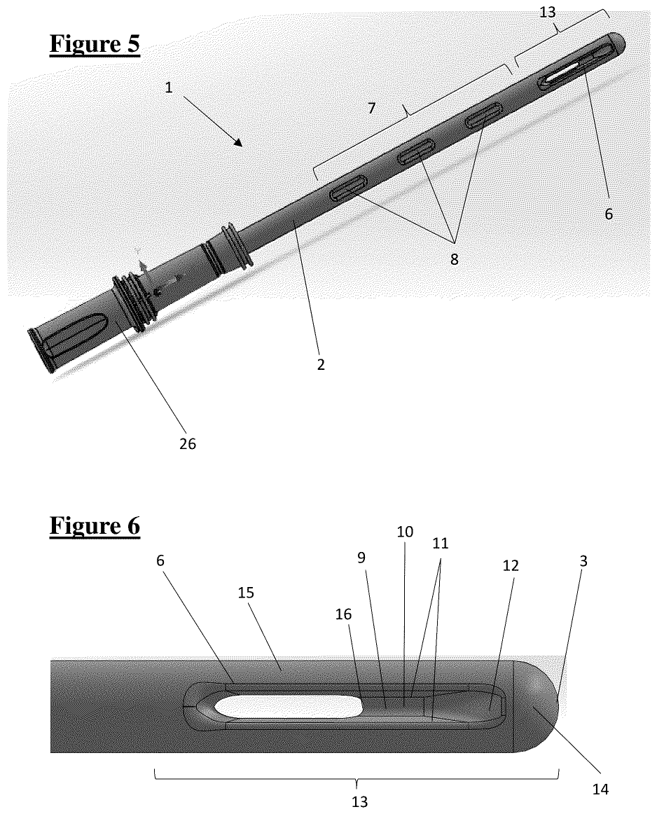

FIG. 5 shows a side view of the catheter of FIGS. 1-4;

FIG. 6 shows an enlarged side view of the inlet aperture of the catheter of FIGS. 1-5;

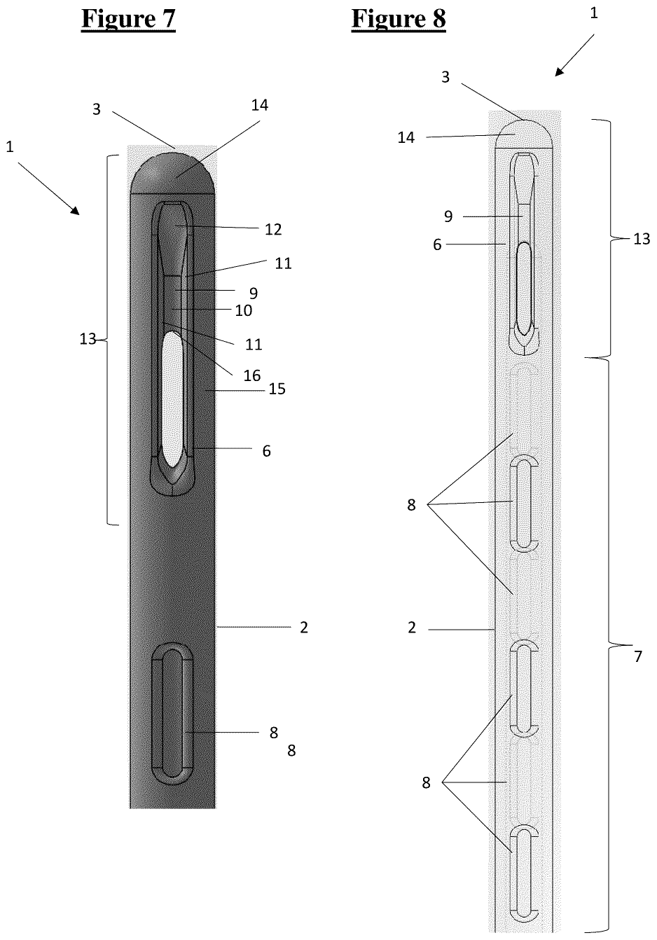

FIG. 7 shows an enlarged view of a proximal section of the inlet aperture and an elongate eyelet of the catheter from the angle of FIG. 1;

FIG. 8 shows a wire frame view of the inlet apertures and plurality of elongate eyelets of the catheter from the angle of FIG. 1;

FIG. 9 shows an axonometric projection from the proximal end of a catheter of the second embodiment;

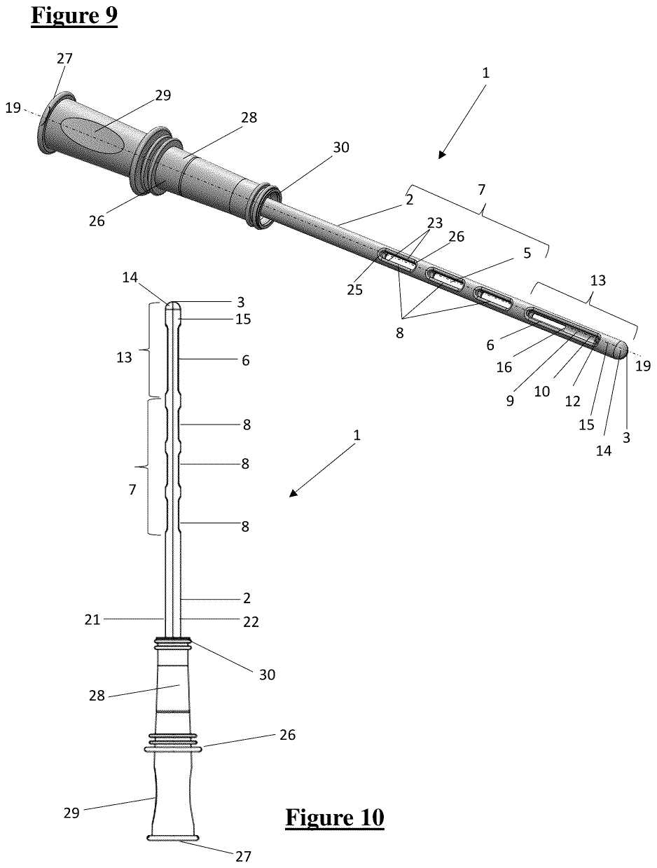

FIG. 10 shows a side view of the catheter of FIG. 9;

FIG. 11 shows a side view of the catheter at 90° to the view of FIG. 10; and

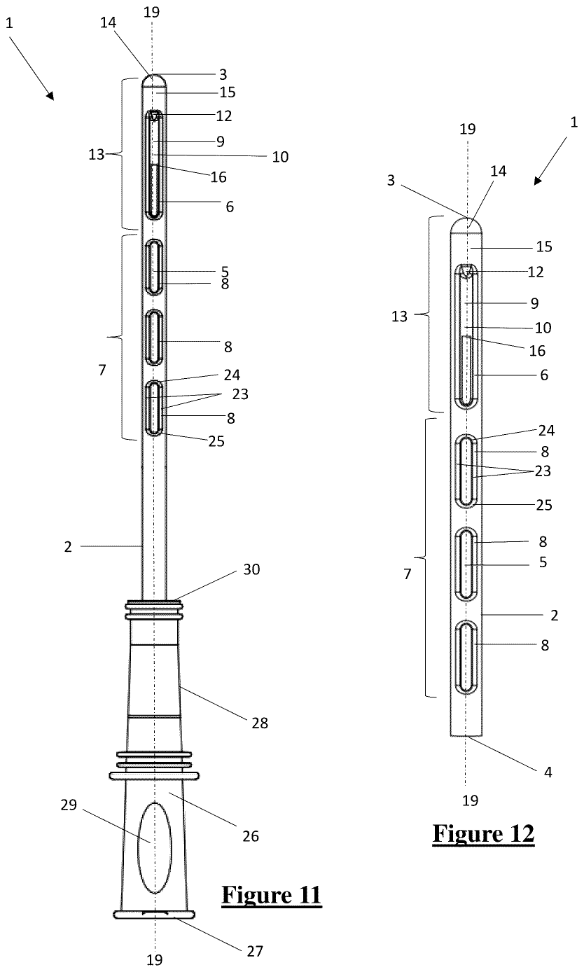

FIG. 12 shows an enlarged side view of the inlet apertures and plurality of elongate eyelets of FIGS. 9-11 from the angle of FIG. 11.

In the following detailed description, numerous specific details are set forth in order to provide a thorough understanding of various embodiments and the inventive concept. However, those skilled in the art will understand that the present invention may be practised without these specific details or with known equivalents of these specific details, that the present invention is not limited to the described embodiments, and that the present invention may be practiced in a variety of alternative embodiments. It will also be appreciated that well known methods, procedures, components, and systems may have not been described in detail.

In the following description, reference to longitudinal should be taken to be in relation to the longitudinal axis of the catheter; the longitudinal axis being the axis extending between the insertion end and the outlet end. Reference to the “transverse cross-section”, or simply “cross-section”, should be taken to be the cross-section which is transverse to the longitudinal axis of the main body, unless otherwise stated. References to distal and proximal made herein should be taken to be in relation to the insertion end of the catheter.

The catheters described herein are primarily female intermittent urinary catheters. However, it will be appreciated that the inventive concepts and features described herein may be applied equally to male intermittent urinary catheters.

Referring to FIGS. 1-8, a first embodiment of an intermittent urinary catheter 1 comprises an elongate main body 2 having a constant outer diameter. The elongate main body 2 has an insertion end 3 for insertion into a urethra and bladder, an outlet end 4 for the outflow of fluid e.g., urine from the catheter, and a lumen 5 extending between the insertion end 3 and the outlet end 4. The elongate main body 2 comprises at least one inlet aperture 6 provided proximal to the insertion end 3. The elongate main body 2 also comprises an external flushing portion 7 which extends between the insertion end 3 and the outlet end 4 and comprises a plurality of elongate eyelets 8 formed through an external surface of the elongate main body 2.

In this particular embodiment, the elongate main body 2 comprises two inlet apertures 6, each having a stadium shape. However, in alternative embodiments it will be understood that a different number of inlet apertures 6 may be provided, for example between one and four inlet apertures 6, and the inlet apertures 6 may be a different shape such as rectangular or oval. The two inlet apertures 6 are offset 180° about the circumference of the elongate main body 2 such that they are diametrically opposed. In this embodiment, the two inlet apertures 6 are not longitudinally offset. However, it will be understood that each inlet aperture 6 may be offset both longitudinally and circumferentially from another. Each inlet aperture 6 in this particular embodiment has a length of 14 mm and a width of 1 mm. However, it will be understood that in alternative embodiments, each inlet aperture 6 may have different lengths and/or widths. Each inlet aperture 6 in this embodiment comprises an angled i.e. chamfered, edge extending fully around the perimeter of the inlet aperture 6, specifically the edge is radiused, to increase comfort of the user during insertion and removal of the catheter 1.

Each inlet aperture 6 comprises a means for directing flow in the form of a separation wall 9 extending distally from the insertion tip 3, internally in the lumen 5, so as to form an open flow channel 9 at the proximal end of each inlet aperture 6. The open flow channel 9 is recessed into the external surface of the elongate main body 2, and in this embodiment has a width of 1 mm and a length of approximately 7 mm. The open flow channel 9 directs urine from the bladder entering through the at least one inlet aperture into the lumen 5 whereby the flow may pass through the lumen, via the external flushing portion 7, and out of the catheter 1. In this particular embodiment, the open flow channel 9 extends over approximately half of the length of the at least one inlet aperture 6. However, a person skilled in the art will understand that the open flow channel 9 may extend over a greater or lesser proportion of the length of the at least one inlet aperture.

The open flow channel 9 comprises a substantially flat base 10 and a pair of substantially flat side walls 11 adjacent to the base 10 seen best in FIGS. 6 and 7. One of the pair of side walls 11 extends along the left hand side of the base and the other side wall 11 extends along the right hand side of the base 10, and both side walls 11 are angled such that the open flow channel 9 has a substantially “U” shaped cross section, shaped to guide a flow of urine from the bladder toward the distal end of the inlet aperture 6 and into the lumen 5. The cross-sectional area of the open flow channel 9 is constant along a major portion. However, toward the proximal end of the open flow channel 9, there is provided an angled portion 12 which assists in directing urine from the blader through the open flow channel 9 and toward the lumen 5.

The open flow channel 9 terminates at a point approximately halfway along the length of the at least one inlet aperture 6. The termination point 16 of the open flow channel has a concavely curved profile which defines the lower portion of the inlet aperture 6. The lower portion of the inlet aperture 6 has a stadium shaped profile defined at the proximal end by the termination point 16 of the open flow channel 9 and at the distal end, by the outline of the inlet aperture 6 formed through an external surface of the elongate main body 2. However, it will be understood that the termination point 16 of the open flow channel 9 may have various profiles such as straight or convexly curved, thereby adjusting the profile of the lower portion of the inlet aperture 6. The lower portion of the first inlet aperture aligns with the lower portion of the second inlet aperture such that the lower portion of the first and second inlet apertures 6 extend fully from a first side of the catheter to a second side of the catheter 1.

The catheter 1 is provided with an insertion portion 13 to facilitate insertion of the catheter 1 into and along the urethra. The insertion portion comprises a rounded tip 14 provided at the insertion end 3 to improve comfort for the user during insertion and a hollow tubular wall 15 in which the at least one inlet aperture 6 is provided. The hollow tubular wall 15 also defines the lumen 5 and comprises the integrally formed open flow channel 9 which is in fluid communication with the lumen 5.

In this particular embodiment, the insertion portion 13 and the at least one inlet aperture 6 are configured to be partially inserted into the bladder. The at least one inlet aperture 6 is configured to be inserted such that at least the open flow channel 9 is positioned in use within the bladder. This means that any urine on the bottom of the bladder may exit the bladder via the inlet aperture which overlaps a junction between the bladder and the urethra to ensure complete bladder emptying.

The elongate main body 2 has a straight, substantially tubular form with an open outlet end 4, and a closed insertion end 3 by virtue of the rounded tip 14. The external surface of the elongate main body 2 forms a wall having a substantially cylindrical shape whose inner surface defines the lumen 5 (seen in FIG. 4). The lumen extends from a first end 17 of the insertion portion 13, specifically from the termination point 16 of the open flow channel 9, where the lumen 5 is in fluid communication with the inlet apertures 6, to a second end 18 provided at the outlet end 4 whereby urine may exit the lumen 5. The lumen 5 has a constant cross section, such that urine entering the at least one inlet aperture 6 passes through the entire length of the lumen 5 with a substantially undisturbed flow having a substantially regular flow rate.

The external flushing portion 7 is configured in use to provide fluid communication between the catheter 1 and the urethra via the plurality of elongate eyelets 8. The plurality of elongate eyelets 8 are formed through the external surface of the elongate main body such that each elongate eyelet 8 enables urine passing through the lumen 5 to contact and flush the walls of the urethra, assisting in reducing the bacteria present. In this particular embodiment there are six elongate eyelets 8, although it will be understood that in alternative embodiments a greater or lesser number of elongate eyelets 8 may be provided.

The plurality of elongate eyelets 8 each have a longitudinal axis 19 which extends parallel to the longitudinal axis of the elongate main body 2. The plurality of elongate eyelets are spaced/offset along the longitudinal axis 19 by the length of approximately one of the plurality of elongate eyelets 8, which in this particular embodiment is approximately 7 mm. Spacing the plurality of elongate eyelets 8 ensures that the structural integrity of the elongate main body 2 is maintained as the presence of the outer surface of the elongate main body 2 between each of the plurality of elongate eyelets 8 maintains the rigidity of the catheter 1 whilst allowing some flexibility enabling the catheter to conform to the shape of the users urethra. This means that the catheter can withstand the pressures exerted by the walls of the urethra during insertion and use without bending and/or buckling.

The plurality of elongate eyelets 8 each have a radial axis which each extend radially with respect to the radial axis of the elongate main body 2. In this embodiment, best seen in FIG. 2, none of the plurality of elongate eyelets 8 directly oppose another of the diametrically opposed plurality of elongate eyelets 8. A person looking through one of the plurality of elongate eyelets 8 would not be able to view another of the plurality of elongate eyelets 8 diametrically opposite and would instead see the internal surface of the elongate main body 2.

In this particular embodiment, the six elongate eyelets 8 are formed through the external surface of the elongate main body 2 along a plane of the longitudinal axis of the elongate main body which extends through the central axis of the elongate main body. The plane intersects the external surface of the elongate main body 2 on a first side of the elongate main body 21 and on a second side of the elongate main body 22 such that the first and second side are diametrically opposed.

With specific reference now to FIGS. 2, 4 and 8, a first series of three elongate eyelets 8 are provided along the first side of the elongate main body 2 at the point where the plane of the longitudinal axis intersects. Each of the three elongate eyelets 8 are the same length and width, and are stadium shaped. In this particular embodiment, each elongate eyelet has a length of 7 mm and a width of 1 mm. However, in alternative embodiments each elongate eyelet 8 may have a different length and/or width to this embodiment, and at least one elongate eyelet may have a different length and/or width to another of the plurality of elongate eyelets. The three elongate eyelets 8 are longitudinally spaced from one another by the approximate length of an elongate eyelet 8 such that there is a solid section of the elongate main body interspersed between the three elongate eyelets which helps to maintain the structural integrity of the catheter 1 whilst still providing some flexibility for the elongate main body 2 to bend. In this particular embodiment, the first inlet aperture 6 is also provided along the first side 21 of the elongate main body 2 at the point where the plane of the longitudinal axis intersects. The elongate eyelet 8 in the first series proximal to the insertion end is longitudinally spaced from the inlet aperture 6 by the approximate length of an elongate eyelet 8.

A second series of three elongate eyelets 8 are provided along the second side 22 of the elongate main body 2 at the point where the plane of the longitudinal axis intersects. The three eyelets of the second series are the same length, width and shape as the first series such that each of the plurality of elongate eyelets is identical. However, in other embodiments it will be understood that at least one of the plurality of elongate eyelets 8 may have a different shape and/or length and/or width to the other elongate eyelets 8. Each of the three eyelets 8 of the second series are longitudinally spaced from one another by the approximate length of an elongate eyelet 8. In this particular embodiment, the second inlet aperture 6 is also provided along the second side of the elongate main body 2 at the point where the plane of the longitudinal axis intersects. The elongate eyelet 8 in the second series proximal to the insertion end is longitudinally spaced from the inlet aperture by approximately 2.5 mm. The first series of elongate eyelets diametrically opposes the second series of elongate eyelets. However, as each series is spaced from the inlet aperture by a differing amount, the first series of elongate eyelets is staggered such that they do not directly oppose the second series; as such a person looking through an elongate eyelet of the first series would not be able to see an elongate eyelet of the second series directly opposite. Instead, such a person would see the internal surface of the elongate main body 2 of the space between the eyelets of the second series.

In this particular embodiment, the first series of elongate eyelets are perfectly longitudinally spaced from the second series of elongate eyelets such that an elongate eyelet is provided on the second side of the elongate main body 2 in the position directly opposing the portion between two elongate eyelets 8 of the first series.

The plurality of elongate eyelets 8 of this particular embodiment each have a pair of parallel long sides 23 connected by a first short side 24 and a second short side 25. The first short side 24 and second short side 25 in this embodiment are both curved to define the curved portions of the overall stadium shape of each elongate eyelet 8. However, it will be understood that in alternative embodiments, the plurality of elongate eyelets 8 may have an alternative shape and/or shapes and as such do not comprise a pair of parallel long sides and a first and second short side. Equally, in alternative embodiments, the first short side 24 and the second short side 25 may not be curved but may instead be straight for example to form a rectangular elongate eyelet 8.

In this particular embodiment, the pair of long sides 23 and the first and second short sides have an angled edge/profile i.e. the edges are chamfered. The angled profile extends fully around the perimeter of each of the plurality of elongate eyelets such that there are no sharp edges and/or corners which may cut or catch a user's skin during insertion and/or removal of the catheter 1.

With reference again to FIGS. 1-8, the outlet end 4 is configured for the attachment of a funnel 26. The funnel 26 forms a substantially conical cavity (not shown) which facilitates the outflow of urine from the catheter 1 via an outlet at an end face of the funnel 27.

The funnel 26 comprises a substantially tubular solid wall portion 28 with a diameter greater than that of the outlet end 4 and external flushing portion 7. The tubular wall 28 defines the conical cavity which extends from the proximal end of the funnel 26 through to the conical cavity. The conical cavity is shaped and configured for the insertion of the outlet end 4 therein with a tight fit. Accordingly, when the funnel 26 is attached to the outlet end 4, a passageway from the external flushing portion 7 is defined by the outlet end 4 and funnel 26 to provide a fluid flow path for an exiting stream of urine from the bladder and urethra, when in use.

The outer surface of the funnel 26 comprises a pair of diametrically opposed grip surfaces 29 provided at the distal end of the funnel 26 and having a substantially flat oval shaped surface. The pair of grip surfaces 29 encourages users to grip the catheter funnel with their thumb and forefinger, at the distal end of the funnel 26, thereby reducing the risk of bacteria from the users fingers entering the urethra and/or bladder during insertion and use of the catheter 1.

The proximal end of the funnel 26 is shaped to direct urine toward the conical cavity to minimise the risk of urine leaking around the funnel 26 and contaminating the users hands. Any urine which has not re-entered the lumen 5 but has instead flowed along the outer surface of the elongate main body 2 is collected at the proximal end of the funnel via the converging portion 30 of the funnel and directed toward the conical cavity where it joins the urine which has flowed out of the outlet end 4 via the lumen 5.

With reference to FIGS. 9-12, a second embodiment of the present invention is shown. The catheter 1 of the second embodiment includes many of the same features as the first embodiment, like features are given like reference numerals, and the detailed description focusses on the differences in the second embodiment.

The differences primarily lie in the open flow channel 9 of the inlet aperture 6 and the positioning of the plurality of elongate eyelets 8. For example, whilst the inlet aperture 6 of the second embodiment is generally the same size and shape as that of the first embodiment, the open flow channel 9 has a slightly different formation. The open flow channel 9 has a substantially flat base 10 recessed into the elongate main body 2, which extends in this manner across the entire width of the inlet aperture 6, until it intersects and is attached to an internal surface of the insertion portion 13 of the elongate main body 2. The open flow channel 9 has a substantially constant cross-sectional area along a major portion.

The open flow channel 9 covers approximately half the length of the inlet aperture 6 and the proximal end of the open flow channel 9 comprises an angled portion 12, similar to the first embodiment. However, the angled portion 12 of the second embodiment is significantly shorter in length than that of the first embodiment. The angled portion has a steeper gradient, attaching the proximal end of the open flow channel 9 to the proximal curved edge of the inlet aperture 6.

The termination point 16 of the open flow channel 9 has a straight profile. In this particular embodiment, there are provided two inlet apertures 6 which are diametrically opposed and directly oppose one another (i.e. they are not longitudinally offset). As they are not longitudinally offset, the base 10 of the open flow channel 9 of the first inlet aperture 6 also forms the base 10 of the open flow channel of the second inlet aperture 6. The second inlet aperture also comprises an angled portion attaching the proximal end of the open flow channel 9 to the proximal curved edge of the second inlet aperture 6. In this way, the first and second inlet apertures 6 are identical.

A first series of three elongate eyelets 8 are provided along the first side of the elongate main body 2 at the point where the plane of the longitudinal axis intersects. The three elongate eyelets 8 of the first series 8 are longitudinally spaced/offset from one another by the approximate length of an elongate eyelet 8, which is 7 mm in this particular embodiment. A second series of three elongate eyelets 8 are provided along the second side of the elongate main body 2 at the point where the plane of the longitudinal axis intersects. The three eyelets of the first series 8 are also longitudinally spaced/offset from one another by the approximate length of an elongate eyelet 8.

In this particular embodiment, the elongate eyelet 8 proximal to the inlet aperture in both the first and second series is longitudinally spaced from the distal end of the first and second inlet apertures 6 respectively by approximately 1.5 mm. In this way each of the elongate eyelets 8 of the first series directly opposes an elongate eyelet of the second series. A person looking through an elongate eyelet of the first series would be able to see an elongate eyelet of the second series directly opposite, and the elongate eyelets would be perfectly aligned. In this way, the catheter 1 of the second embodiment is symmetrical about the plane upon which the elongate eyelets are formed as well as being symmetrical about the plane perpendicular to this which also passes through the central axis of the elongate main body 2.

The catheter 1 of the embodiments of FIGS. 1-8 and 9-12 are both formed via injection moulding. However, in alternative embodiments other suitable methods of manufacture may be used. For example, the elongate main body 2 may be extruded and the at least one inlet aperture and/or plurality of elongate eyelets 8 may subsequently be punched into an external surface thereof.

The catheter 1 may be inserted by a user into the urethra until the inlet aperture 6 is at least partially inserted into the bladder. Urine in the bladder may then flow from the bladder into the inlet aperture 6 via the open flow channel 9, and into the lumen 5. The urine flows through the lumen 5 to the outlet end 4 where it may be discharged into a suitable receptacle. As the urine flows through the lumen 5, flushing of the urethral walls is enabled by the plurality of elongate eyelets 8 which enable the urine to contact the urethral walls and flush away bacteria to minimise the risk of infection.

The catheter of the embodiment shown in FIGS. 1-8 having each of the plurality of elongate eyelets 8 perfectly longitudinally spaced apart from one another provides a fluid flow path of relatively constant cross-sectional area through which the urine may flow. In this way, the urine may exit the catheter 1 in an efficient manner with minimal turbulence. This enables rapid draining of the bladder. On the other hand, in the embodiment of FIGS. 9-12, whereby the plurality of elongate eyelets 8 directly oppose one another, the cross-sectional area of the flow path is more changeable than that of the first embodiment. Although this means that the flow of urine passing therethrough is more turbulent and so less efficient, the turbulent flow encourages greater flushing of the walls of the urethra through the plurality of elongate eyelets 8. This is particularly advantageous in minimising the build-up of bacteria on the walls of the urethra.

In some embodiments, the catheter is formed of a material of the group comprising: polyvinyl chloride, polytetrafluoroethylene, polyolefins, latex, silicones, synthetic rubbers, polyurethanes, polyesters, polyacrylates, polyamides, thermoplastic elastomeric materials, styrene block copolymers, polyether block amide, thermoplastic vulcanizates, thermoplastic copolyesters, thermoplastic polyamides, and water disintegrable or enzymatically hydrolysable material, or combinations, blends or co-polymers of any of the above materials.

In preferred embodiments, the catheter is formed of a material of the group comprising: polyolefins, polyesters, polyacrylates, polyamides, thermoplastic elastomeric material, polyether block amide, thermoplastic vulcanizates, thermoplastic copolyesters, thermoplastic polyamides, fluororubber, and water disintegrable or enzymatically hydrolysable material or combinations, blends or co-polymers of any of the above materials.

In some embodiments, said water disintegrable or enzymatically hydrolysable material comprises a material of the group comprising: polyvinyl alcohol, extrudable polyvinyl alcohol, polyacrylic acids, polylactic acid, polyesters, polyglycolide, polyglycolic acid, poly lactic-co-glycolic acid, polylactide, amines, polyacrylamides, poly(N-(2-Hydroxypropyl) methacrylamide), starch, modified starches or derivatives, amylopectin, pectin, xanthan, scleroglucan, dextrin, chitosans, chitins, agar, alginate, carrageenans, laminarin, saccharides, polysaccharides, sucrose, polyethylene oxide, polypropylene oxide, acrylics, polyacrylic acid blends, poly(methacrylic acid), polystyrene sulfonate, polyethylene sulfonate, lignin sulfonate, polymethacrylamides, copolymers of aminoalkyl-acrylamides and methacrylamides, melamine-formaldehyde copolymers, vinyl alcohol copolymers, cellulose ethers, poly-ethers, polyethylene oxide, blends of polyethylene-polypropylene glycol, carboxymethyl cellulose, guar gum, locust bean gum, hydroxypropyl cellulose, vinylpyrrolidone polymers and copolymers, polyvinyl acetate, polyvinyl pyrrolidone-carboxymethyl cellulose, carboxymethyl cellulose shellac, copolymers of vinylpyrrolidone with vinyl acetate, hydroxyethyl cellulose, gelatin, poly-caprolactone, poly(p-dioxanone), or combinations, blends or co-polymers of any of the above materials. In some preferred embodiments, the catheter is formed of a polyolefin material, especially polyethylene and/or polypropylene. In some preferred embodiments, the catheter is formed of a thermoplastic elastomeric material. In some preferred embodiments the catheter may be made from a polyolefin based synthetic thermoplastic polyolefin elastomer (TPE) containing a hydrophilic additive. In some preferred embodiments the catheter may be made from a polymer mixture comprising a first polymer and a second polymer, wherein the first polymer is a thermoplastic or thermo-curing polymer, and the second polymer is an amphiphilic block copolymer possessing both hydrophilic and lipophilic properties. A possible suitable polymer mixture is described and disclosed in EP2493521A1.

The one or more embodiments are described above by way of example only. Many variations are possible without departing from the scope of protection afforded by the appended claims.

1. An intermittent urinary catheter comprising:
an elongate main body having an insertion end for insertion into a urethra and bladder, an outlet end for the outflow of fluid from the catheter, and a lumen extending between the insertion end and the outlet end,
the elongate main body further comprising at least one inlet aperture proximal to the insertion end, and an external flushing portion extending along a portion of the elongate main body between the insertion end and the outlet end,
wherein the external flushing portion comprises a plurality of elongate eyelets formed through an external surface of the elongate main body.

2. The intermittent urinary catheter of claim 1 wherein the at least one inlet aperture comprises an open flow channel provided in the external surface of the elongate main body.

3. The intermittent urinary catheter of claim 2 wherein the open flow channel extends over at least one half of the length of the at least one inlet aperture.

4. The intermittent urinary catheter of any preceding claim wherein the at least one inlet aperture is in fluid communication with the external flushing portion via the lumen.

5. The intermittent urinary catheter of any preceding claim wherein the at least one inlet aperture is stadium shaped.

6. The intermittent urinary catheter of any preceding claim wherein the plurality of elongate eyelets have a longitudinal axis which extends parallel to the longitudinal axis of the elongate main body.

7. The intermittent urinary catheter of claim 6 wherein the plurality of elongate eyelets are spaced along the longitudinal axis of the elongate main body.

8. The intermittent urinary catheter of any preceding claim wherein at least one of the plurality of elongate eyelets does not directly oppose any other of the plurality of elongate eyelets.

9. The intermittent urinary catheter of any preceding claim wherein at least one of the plurality of elongate eyelets directly opposes another of the plurality of elongate eyelets.

10. The intermittent urinary catheter of any preceding claim wherein at least one of the plurality of elongate eyelets partially opposes another of the plurality of elongate eyelets.

11. The intermittent urinary catheter of any preceding claim wherein the plurality of elongate eyelets are provided along a plane of the longitudinal axis of the elongate main body.

12. The intermittent urinary catheter of any preceding claim wherein a first series of elongate eyelets is formed through a first side of the elongate main body and a second series of elongate eyelets is formed through a second side of the elongate main body.

13. The intermittent urinary catheter of claim 12 wherein the first side of the elongate main body and the second side of the elongate main body are diametrically opposed.

14. The intermittent urinary catheter of claim 12 or 13 wherein each series comprises three elongate eyelets.

15. The intermittent urinary catheter of claim 14 wherein none of the elongate eyelets of the first series directly opposes another of the elongate eyelets of the second series.

16. The intermittent urinary catheter of claim 14 wherein each of the elongate eyelets of the first series directly opposes an elongate eyelet of the second series.

17. The intermittent urinary catheter of any preceding claim wherein at least one of the plurality of elongate eyelets has a width in the range of 0.8 mm to 1.2 mm.

18. The intermittent urinary catheter of any preceding claim wherein at least one of the plurality of elongate eyelets has a width of 1 mm.

19. The intermittent urinary catheter of any preceding claim wherein at least one of the plurality of elongate eyelets has a length in the range of 2.5 mm to 8 mm.

20. The intermittent urinary catheter of any preceding claim wherein at least one of the plurality of elongate eyelets has a length of 7 mm.

21. The intermittent urinary catheter of any preceding claim wherein the plurality of elongate eyelets is stadium shaped.

22. The intermittent urinary catheter of any preceding claim wherein the intermittent urinary catheter is a female intermittent urinary catheter.
